(19) United States
(12) Patent Application Publication
(10) Pub. No.: US 2003/0125677 A1
(43) Pub. Date: Jul. 3, 2003

(54) Medical needle assemblies

(75) Inventors: Jon D. Swenson, Wayne, NJ (US); Richard James Caizza, Vernon, NJ (US); Kirk D. Swenson, North Caldwell, NJ (US); Alfred Wesley Prais, Hewitt, NJ (US); Paul G. Alchas, Wayne, NJ (US)

Correspondence Address: Kirk M. Miles, Esq., WEBB ZIESENHEIM LOGSDON ORKIN & HANSON, P.C., 700 Koppers Building, 436 Seventh Avenue, Pittsburgh, PA, 15219-1818, US

(73) Assignee: Becton, Dickinson and Company

(21) Appl. No.: 10/141,174

(22) Filed: May 9, 2002

Related U.S. Application Data
(60) Provisional application No. 60/344,126, filed on Dec. 28, 2001.

(51) Int. Cl. A61M 5/32

(52) U.S. Cl. 604/263; 128/919

(57) ABSTRACT

A shieldable, single-use unit dose needle assembly for administering a unit dose of a vaccine includes a unit dose needle attached to a base with a sleeve slidably attached thereto. The sleeve includes a guide slot that extends through the tubular sleeve wall. The base includes a tab disposed on an outer surface and extends through and beyond the guide slot. The unit dose needle includes a base end attached to the base and an opposed prong end configured to hold a unit dose of a vaccine. The needle assembly is configured such that movement of the tab through the guide slot enables an extension and a retraction movement of the needle between an extended position with the needle extending from the distal end of the sleeve and a retracted position with the needle contained within the internal sleeve opening.

### CROSS-REFERENCE TO RELATED APPLICATIONS

This application claims priority from U.S. Provisional Patent Application Serial No. 60/344,126, filed Dec. 28, 2001, entitled “Bifurcated Needle Assembly with Needle Shielding Provisions.”

### BACKGROUND OF THE INVENTION

1. Field of the Invention

The present invention relates to needles for use in medical procedures and, in particular, to safety shielded needles and needle assemblies for use in medical procedures.

2. Description of Related Art

Bifurcated or forked end needles are well-known for providing a simple and effective means for a doctor to administer a vaccine. During use, the bifurcated tip of the bifurcated needle is put into contact with either a dried or liquid substance, which adheres to the bifurcated needle tip. The bifurcated needle tip is then put into contact with the skin of the patient who is being administered the vaccination. The skin is either scratched or pierced with the needle tip so that the vaccination material may be absorbed into the skin of the patient. An alternative method of delivering the vaccination includes placing a drop of the vaccine onto the skin of the patient and contacting the skin of the patient with the bifurcated needle tip through the drop of vaccination. Alternatively, although not ideal, a standard pointed needle tip may also be used when the drop of vaccination is applied directly to the skin of the patient.

The bifurcated needle is considered a significant medical advancement because it has allowed more people to be vaccinated with less serum. This has been especially important for those living in less developed areas because of the efficient and easy to use design, as well as the ease of replication.

Vaccination effectiveness, however, is reduced if the bifurcated needle is reused too many times. Moreover, reuse of such vaccination needles exposes patients to the risk of transmission of infectious diseases through percutaneous contact through the skin. Additionally, medical care workers using traditional vaccination needles are at an increased risk of exposure to infectious diseases due to the design of such needles, which makes them difficult to handle, as well as due to the repeated use of such needles.

In particular, bifurcated needles used to administer vaccinations are not traditionally sterilized or packaged in a single-use container that would enable convenient storage and subsequent use. Additionally, such needles have traditionally been difficult to handle in that they typically do not include a hub attached to the opposite end of a needle from the tip, and do not typically include any sort of shield for protection from the needle prior to and during use.

For example, U.S. Pat. No. 3,194,237 to Rubin discloses a vaccination needle having a main shank with a pair of prongs at one end that define a slot of predetermined length, width, and depth therebetween to hold an amount of liquid by capillary action. The shank of the needle is of sufficient length so that the non-prong end will function as a handle. U.S. Pat. No. 3,948,261 to Steiner discloses a reusable unit dose container for vaccines contained within a rigid receptacle, with a compressible closure for supporting a bifurcated needle bearing dried vaccine. The closure is adapted to support the needle in the container during a lyophilizing process while liquid vaccine is dried on the needle. The closure has grooves which permit the vaporized liquid from the vaccine to be withdrawn from the receptacle during lyophilizing, and can further seal the container.

Numerous devices have been developed in the medical field for shielding needles after use. Many of these devices are somewhat complex and costly. In addition, many of these devices are cumbersome to use in performing procedures. Furthermore, some of the devices are so specific that they preclude use of the device in certain procedures or with certain devices and/or assemblies.

For example, U.S. Pat. No. 6,093,170 to Hsu et al. discloses a safety syringe that includes a barrel having two trapezoidal-shaped tabs, a sleeve with a guide slot and limit slots extending from the circumferential surface at the two ends of the guide slot, and an insertion section formed at the inside end of the limit slot. The tabs are utilized to engage and the insertion section enables the positioning of the barrel and the sleeve. U.S. Pat. No. 5,591,138 to Vaillancourt discloses a multi-component needle assembly that includes a V-shaped slot provided at the rear of a guide slot so that a retractable sheath can be locked in a retracted position. A cam surface is provided at the front of the guide slot to direct the projection on the sheath into a position to lock of the sheath in an extended position.

In view of the foregoing, there exists a need for a safety assembly for use with a unit dose vaccination needle that is easily manufactured, that is simple to use, that is easily sterilized and maintained in a sterile condition until used, that can be safely disposed of, and that does not interfere with normal practices of bifurcated needle use.

### SUMMARY OF THE INVENTION

The present invention is directed to a shieldable, single-use needle assembly for administering a unit dose of a vaccine. The assembly includes a unit dose needle attached to a base, with a sleeve slidably attached thereto. The sleeve includes a tubular wall defining an internal sleeve opening that extends between a proximal end and an opposed distal end of the sleeve. The sleeve further includes a guide slot that extends through the tubular wall and into the internal sleeve opening. The guide slot includes a longitudinal extent extending longitudinally along the sleeve, a proximal limit extent disposed at a first end of the guide slot adjacent to the proximal end of the tubular wall, and a distal limit extent disposed at a second end of the guide slot adjacent to the distal end of the tubular wall. The proximal and distal limit extents extend along a circumferential surface of the tubular wall. The base includes a tab disposed on an outer surface thereof. The base is contained within the internal sleeve opening with the tab extending through and beyond the guide slot. The unit dose needle includes a base end attached to the base and an opposed prong end configured to hold a unit dose of a vaccine. The needle assembly is configured such that movement of the tab through the guide slot between the proximal limit extent and the distal limit extent provides a guiding action that enables an extension and a retraction movement of the needle between an extended position, with the needle extending from the distal end of the sleeve, and a retracted position, with the needle contained within the internal sleeve opening. The proximal and distal limit extents fix the position of the needle between the extended position and the retracted position.

In a further embodiment, the present invention is directed to a shieldable, single-use needle assembly for administering a unit dose of a vaccine, which includes a base, a unit dose needle having a base end attached to the base and an opposed prong end configured to hold a unit dose of a vaccine, a sleeve including a tubular wall defining an internal sleeve opening extending between a proximal end and an opposed distal end, and a guiding element interengaged between the sleeve and the base. The sleeve coacts axially with the base between a prong exposed position and a prong encapsulated position, and the guiding element provides limited axial movement of the sleeve with respect to the base. The guiding element may be a tab extending from the base into a slot in the sleeve, and the slot may extend through the sleeve, with the tab acting as a means to manually move the base and unit dose needle into the sleeve. Also, the base may include a handle extension extending beyond the proximal end of the sleeve.

In another embodiment, the present invention includes a shieldable, single-use needle assembly for administering a unit dose of a vaccine including a base, a unit dose needle, a tip guard, and a tether element. The unit dose needle includes an axis, a base end attached to the base and an opposed prong end configured to hold a unit dose of a vaccine. The tip guard is slidable along the unit dose needle axis from a first position adjacent the base to a second position encapsulating the unit dose needle prong end. The tether element has a base end integral with the base, a guard end integral with the tip guard, and a hinge between the base end and the guard end. The tether element further includes a forward member delimited by the guard end and the hinge which is flexibly pivotable with respect to the guard, a rearward end delimited by the base end and the hinge which is flexibly pivotable with respect to the base. The tether element has an angle between the forward and rearward members, wherein forward movement of the hinge enlarges the angle and provides direct forward movement of the guard with respect to the base.

### BRIEF DESCRIPTION OF THE DRAWINGS

FIG. 1 is an exploded perspective view of a shieldable single-use needle assembly of the present invention;

FIG. 2 is a perspective view of the shieldable needle assembly of FIG. 1 shown with the needle in a retracted position;

FIG. 3 is a perspective view of the shieldable needle assembly of FIG. 1 shown with the needle in an extended position;

FIG. 4 is a side-sectional view of the shieldable needle assembly as shown in FIG. 2 with the needle in a retracted position;

FIG. 5 is a side-sectional view of the shieldable needle assembly as shown in FIG. 3 with the needle in an extended position;

FIG. 6 is an exploded cross-sectional view of a unit dose needle and a base in an alternate embodiment of the present invention;

FIG. 7 is a perspective view of a needle and a base in a further embodiment of the present invention;

FIG. 8 is a side-sectional view of a needle and a base with a handle extension in a further embodiment;

FIG. 9 is a perspective view of a shieldable needle assembly in a further embodiment of the present invention including ribs on the shield;

FIG. 10 is a side-sectional view of a shieldable needle assembly in yet a further embodiment of the present invention with the needle in an extended position;

FIG. 11 is a side-sectional view of the shieldable needle assembly of FIG. 10 with the needle in a retracted position;

FIG. 12 is a side-sectional view of a shieldable needle assembly in a further embodiment of the present invention including a release latch;

FIG. 13 is a perspective view of a shieldable needle assembly in accordance with a further embodiment of the present invention including a telescoping needle clip; and

FIG. 14 is a cross-sectional view of the assembly of FIG. 13.

### DETAILED DESCRIPTION

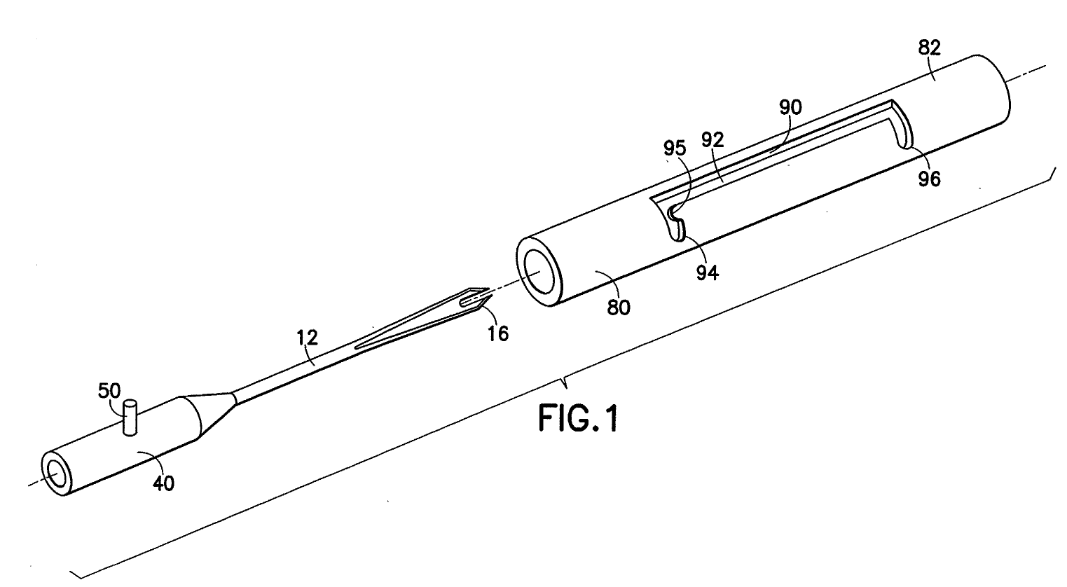

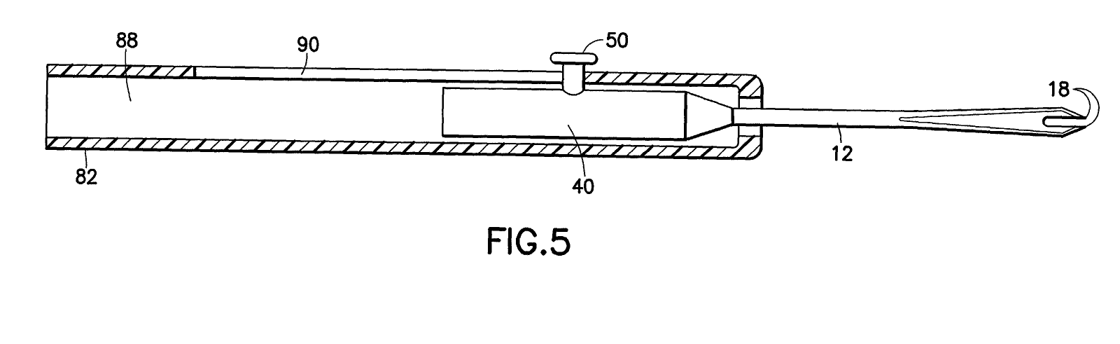

Referring to the drawings in which like reference characters refer to like parts throughout the several views thereof, FIGS. 1-5 depict a shieldable single-use unit dose needle assembly 10 in accordance with the present invention and the related features. The unit dose needle assembly 10 is intended for use for the administration of vaccines applied to or through the skin of the patient, and is intended as a single-use vaccination needle assembly including features to maintain sterility of the needle during packaging and to provide ease of use for the medical practitioner, as will be described in more detail herein.

The needle assembly 10 generally includes an external shield in the form of a tubular hollow sleeve 80, with a base 40 extending within the sleeve 80, and with a unit dose needle 12 extending from the base 40. As will be described in more detail herein, the sleeve 80 and the base 40 are axially movable or displaceable with respect to each other, thereby causing the unit dose needle 12 to move between a retracted position contained entirely within sleeve 80 and an extended position, extending from sleeve 80.

More particularly, as shown in FIGS. 1 and 3-5, the needle assembly 10 includes a unit dose needle for administering a unit dose of a vaccine, such as unit dose needle 12. While needle assembly 10 is described herein in terms of a preferred embodiment including a unit dose needle 12 as the unit dose needle, needle assembly 10 may include any unit dose needle capable of administering a unit dose of a vaccine, such as in a dry powder or liquid form, as is well-known in the art.

The unit dose needle 12 includes a proximal end 14, and an opposed distal end 16. Unit dose needle 12 is provided with two sharp prongs 18 positioned at an opposed distal end 16 of the needle. The prongs 18 are separated by a U-shaped or V-shaped channel 20 configured to hold a unit dose of vaccine. The prongs 18 are intended to penetrate or abrade the skin of the patient to administer the vaccine disposed in the U-shaped channel 20. Unit dose needle 12 may be constructed of any material known in the art, such as metal or plastic, and is desirably constructed of a medical grade surgical steel.

The vaccine may be in any suitable physical form. Suitable physical forms for the vaccine include, but are not limited to, liquids, such as solutions, emulsions, and dispersions, or dry powders. Typically, the vaccine will be in a liquid form. Moreover, the vaccine is desirably associated with needle assembly 10 during storage, and may therefore be contained within the U-shaped channel 20 during storage. Alternatively, the vaccine may be provided as a separate component, with unit dose needle 12 being contacted with the vaccine after removing needle assembly 10 from its associated packaging just prior to use of the needle for administration of the vaccine.

Needle assembly 10 further includes a base 40, which may be integrally formed with unit dose needle 12, or separately attached thereto. Base 40 includes a generally cylindrical housing 42 extending from a proximal end 44 to a distal end 46. Base 40 further includes a guide element such as a tab 50 disposed on an outer surface of the housing 42 of base 40. Tab 50 may be integral with housing 42 or alternately may be separately attached such as by a threaded screw means.

Needle assembly 10 further includes sleeve 80 extending about base 40 in a concentrical manner, thereby enclosing and containing base 40. In particular, sleeve 80 includes a generally tubular wall forming a hollow housing 82 extending between a proximal end 84 and an opposed distal end 86 and defining an internal sleeve opening 88 extending therethrough. Sleeve 80 further includes a guide slot 90 extending through the wall of housing 82 into the internal sleeve opening 88. The guide slot 90 includes a longitudinal extent 92 extending longitudinally along the housing 82 of sleeve 80, a proximal limit extent 94 disposed at a first end of the guide slot 90 adjacent the proximal end 84 of the sleeve 80, and a distal limit extent 96 disposed at a second end of the guide slot 90 adjacent the distal end 86 of the sleeve 80. The proximal and the distal limit extents 94, 96 extend circumferentially along a partial circumferential surface of the housing 82, as shown in FIG. 1.

Sleeve 80 provides protection to personnel from needle sticks prior to movement of unit dose needle 12 between the retracted position and the extended position, as well as after unit dose needle 12 has been moved to the retracted position after use thereof.

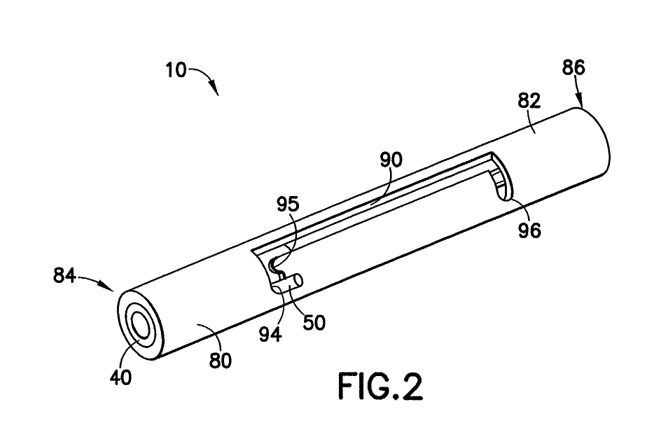

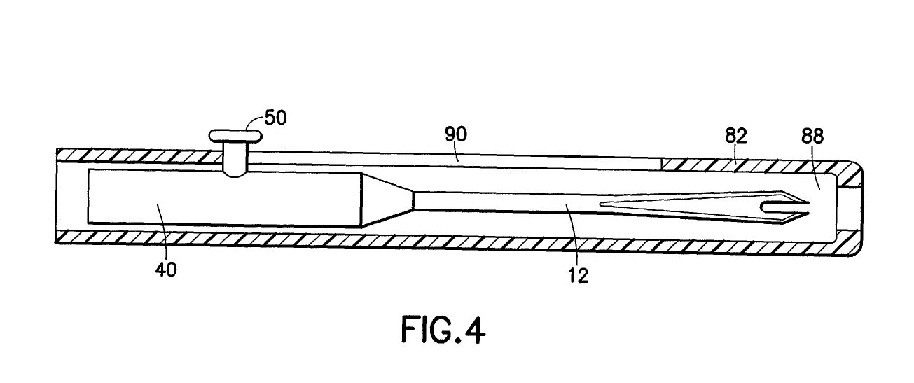

As noted, base 40 is contained within the internal sleeve opening 88 of sleeve 80, when the unit dose needle is in the retracted position or the extended position. For example, as shown in FIGS. 2 and 4, the overall length of the base 40 and unit dose needle 12 extending therefrom is desirably slightly less than the overall length of sleeve 80, such that base 40 and unit dose needle 12 are maintained entirely within internal sleeve opening 88 of sleeve 80 when unit dose needle 12 is retracted within sleeve 80. Also, when the unit dose needle 12 is in the extended position, base 40 is contained entirely within sleeve 80, with no portion of base 40 exposed from proximal end 84 of sleeve 80. With base 40 extending entirely within sleeve 80, there is no portion of base 40 for a user to grasp onto in order to axially move base 40 within sleeve 80 to expose unit dose needle 12 to the extended position or to retract unit dose needle 12 to the retracted position. However, as shown in FIGS. 2-5, tab 50 of base 40 extends through and beyond the guide slot 90 of sleeve 80. As such, tab 50 can be moved through the guide slot 90 to effect movement of base 40 and, therefore, corresponding movement of unit dose needle 12.

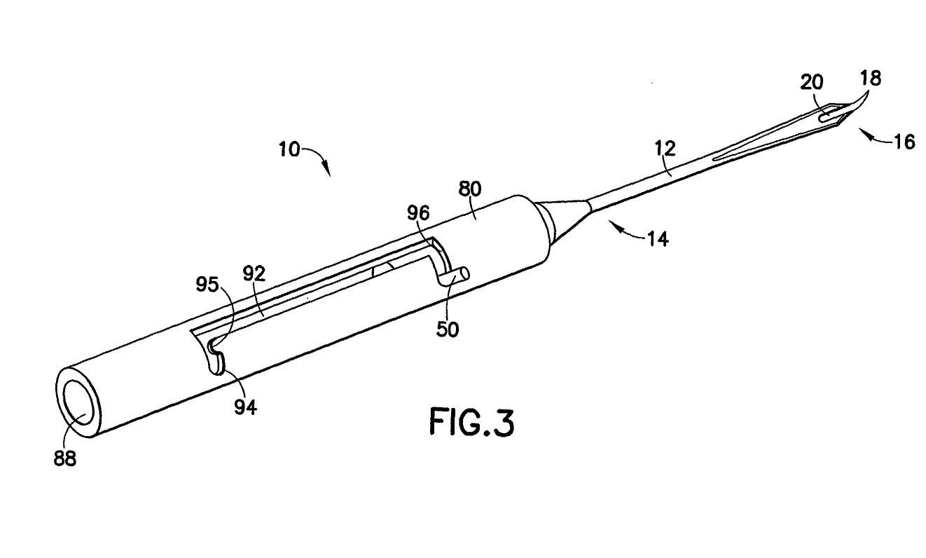

More particularly, a user can grasp needle assembly 10 between a thumb and finger at opposing sides of housing 82 of sleeve 80. Since tab 50 extends through and beyond guide slot 90, the user's index finger can be used to manipulate tab 50. Tab 50 is capable of moving through the guide slot 90 between the proximal limit extent 94 and the distal limit extent 96, which provides a guiding action that enables an extension and a retraction movement of the needle between an extended position as shown in FIG. 3, with the unit dose needle 12 extending from the distal end 86 of the sleeve 80, and a retracted position as shown in FIG. 2, with the unit dose needle 12 contained within the internal sleeve opening 88. As such, unit dose needle 12 can be moved between the extended position and the retracted position with one handed operation of needle assembly 10.

The proximal limit extent 94 and the distal limit extent 96 fix the position of the unit dose needle 12 between the extended position and the retracted position. Desirably, a locking mechanism is provided, such as a lip 95 extending along a portion of the tubular wall of housing 82 within proximal limit extent 94 and distal limit extend 96, thereby fixing the position of the tab within guide slot 90, and providing a tactile feel to the user that the tab 50 has been moved within the guide slot 90 between proximal limit extent 94 and distal limit extend 96.

It is further contemplated that the arrangement of the tab and the slot can be reversed, such that the sleeve 80 includes a tab, and the base 40 includes a guide slot for engagement therewith. As such, the tab of the sleeve 80 can slide within the guide slot of the base 40 in a similar manner as discussed above.

In use, needle assembly 10 may be packaged in sterile packaging, and is removed from the packaging for use. Either a film-based blister package may be used or alternatively a rigid hardpack type of packaging involving thermoplastic shields may be utilized. Needle assembly 10 may be provided as shown in FIG. 2, including unit dose needle 12 retracted within sleeve 80. To prepare for use, unit dose needle 12 is placed in the extended position, by grasping sleeve 80 at opposing sides thereof, and by manipulating tab 50 through guide slot 90, from proximal limit extend 94, along longitudinal extent 92, to distal limit extent 96, to extend unit dose needle 12 from sleeve 80. Alternatively, needle assembly 10 may be provided as depicted in FIG. 3, with unit dose needle 12 extending from sleeve 80, and may further include a separate packaging cover (not shown) covering the unit dose needle 12.

With unit dose needle 12 extending from sleeve 80, needle assembly 10 can be used to administer an appropriate vaccine. For example, a unit dose of a vaccine contained within U-shaped channel 20 may be administered percutaneously to the patient by way of unit dose needle 12. The unit dose of the vaccine may be contained within U-shaped channel 12 during packaging, or the unit dose of the vaccine may be placed within U-shaped channel 20 immediately prior to administration. The vaccination may be administered through several applications to the patient with the same assembly.

After administration of the vaccine is complete, the user grasps sleeve 80 at opposing sides thereof, and manipulates tab 50 through guide slot 90 in reverse manner, from distal limit extent 96, along longitudinal extent 92, to proximal limit extend 94, thereby retracting unit dose needle 12 within sleeve 80. Needle assembly 10 is safely shielded, and can be properly disposed of.

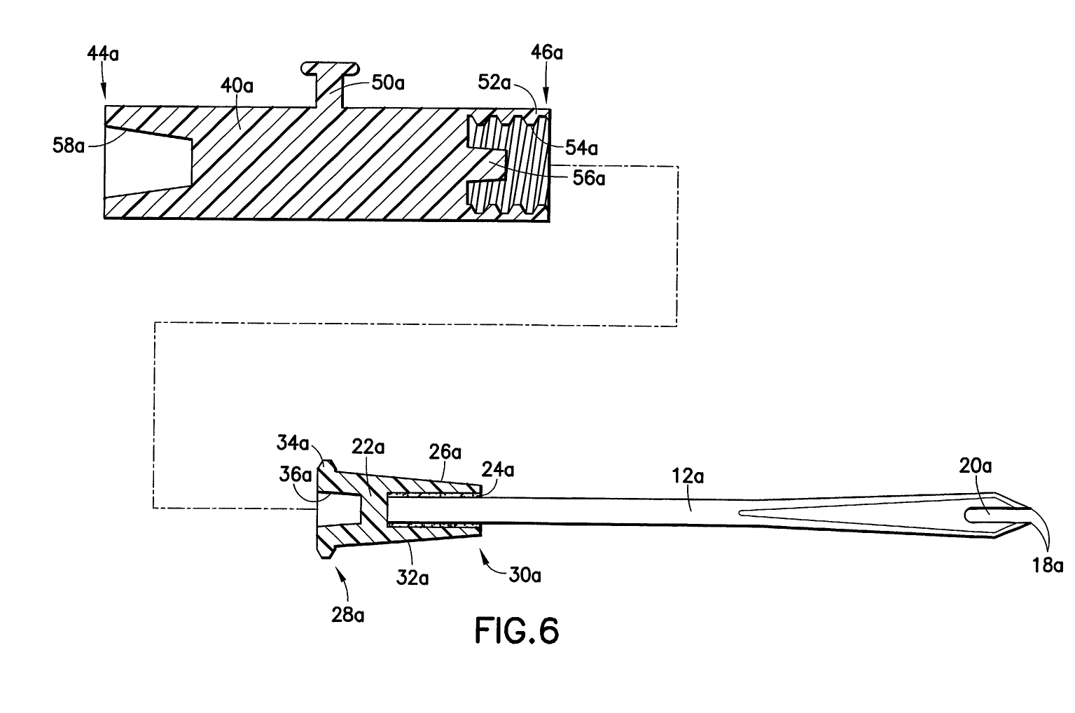

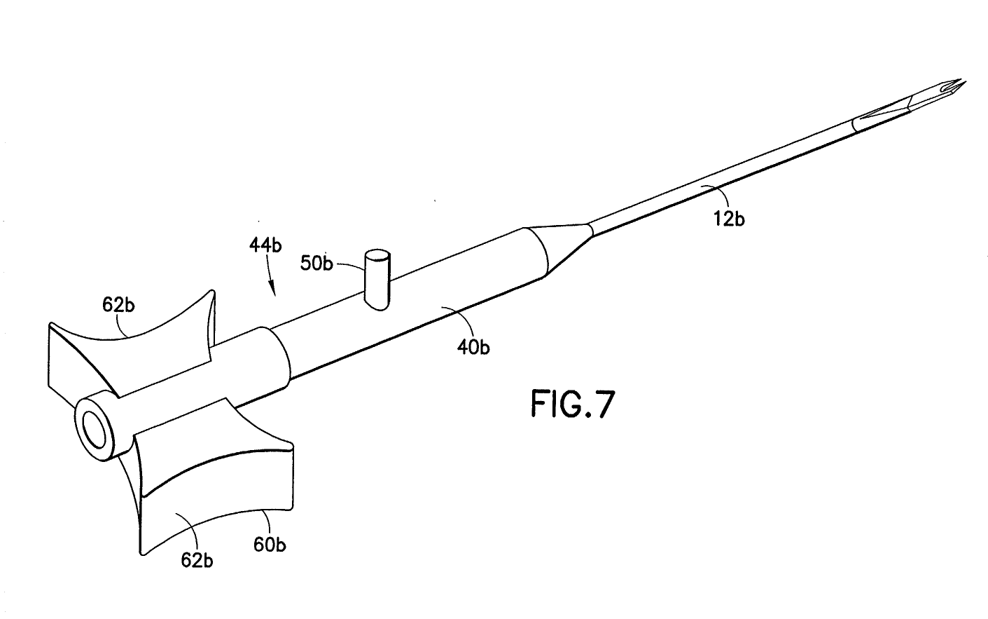

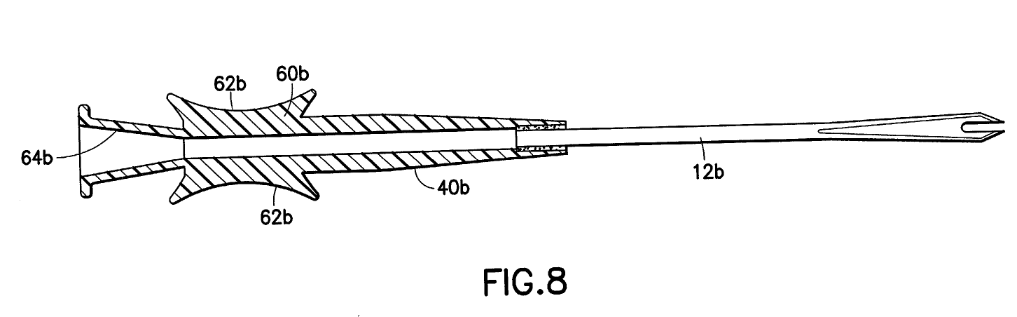

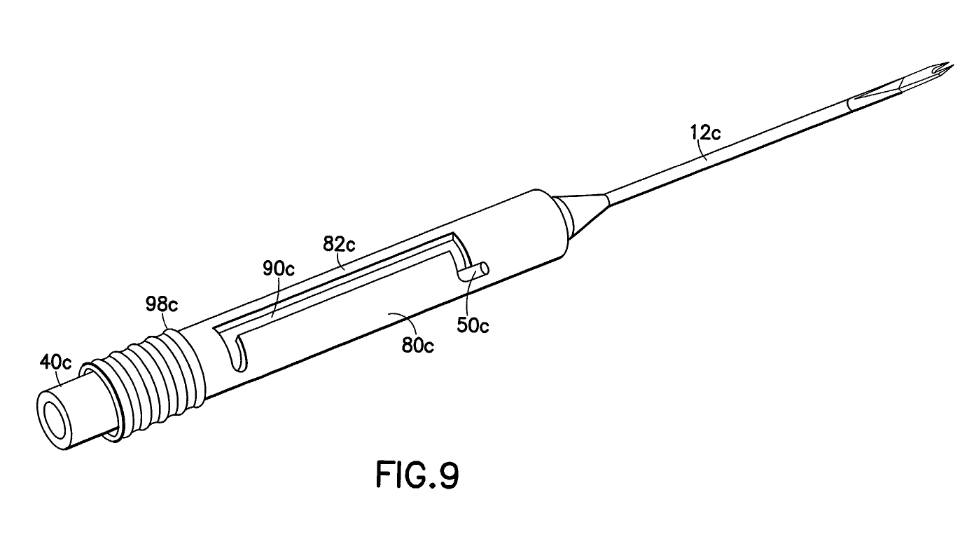

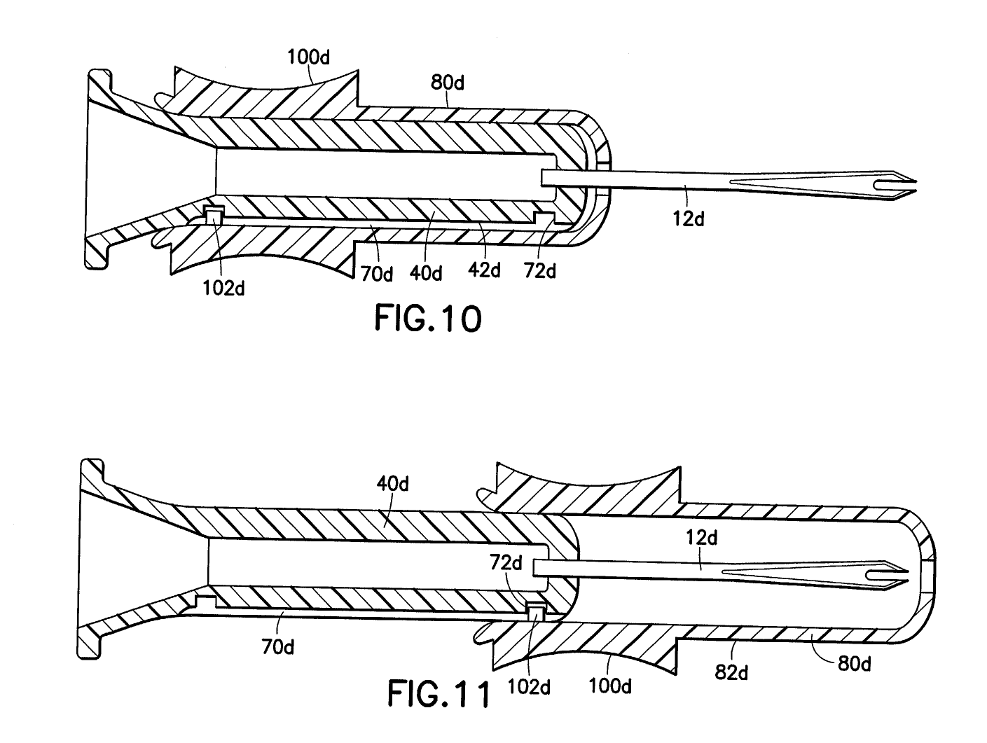

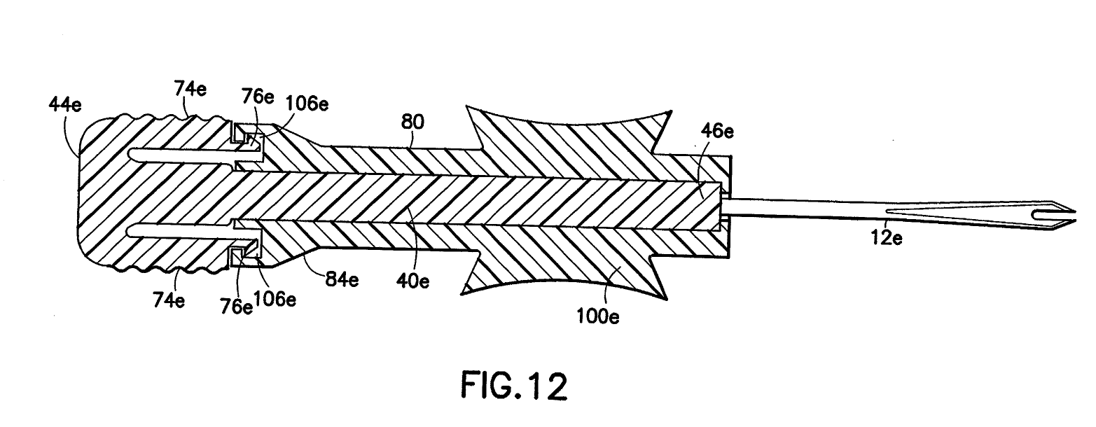

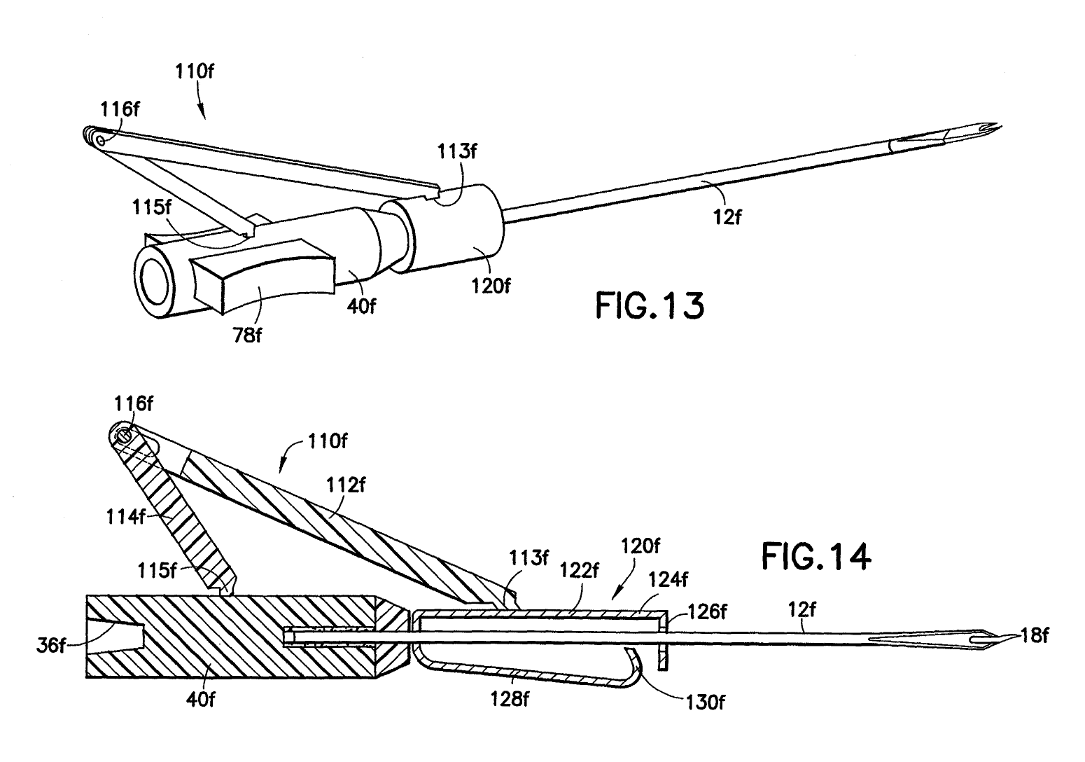

FIGS. 6-14 depict further embodiments of the present invention that include many components which are substantially identical to the components of FIGS. 1-5. Accordingly, similar components performing similar functions will be numbered identically to those components of FIGS. 1-5, except that a suffix “a” will be used to identify those similar components in FIG. 6; a suffix “b” will be used to identify those similar components in FIGS. 7 and 8; a suffix “c” will be used to identify those similar components in FIG. 9; a suffix “d” will be used to identify those similar components in FIGS. 10 and 11; a suffix “e” will be used to identify those similar components in FIG. 12; and a suffix “f” will be used to identify those similar components in FIGS. 13 and 14.

In the alternate embodiment shown in FIG. 6, unit dose needle 12a may further include a hub 22a fixedly attached to the proximal end 14a of unit dose needle 12a, such as through an adhesive joint 24a. Adhesive joint 24a may be provided through any adhesive capable of fixedly attaching or adhering unit dose needle 12a to hub 22a, such as an epoxy or equivalent adhesive. Hub 22a includes a hub housing 26a including a proximal end 28a and a distal end 30a. Desirably, distal end 30a of hub 22a includes an internal bore having an internal diameter of approximately the same size as or a slightly larger size than the outer diameter of the proximal end 14a of unit dose needle 12a, for accommodating and fixedly adhering unit dose needle 12a within such an internal bore of hub 22a.

The hub 22a may further include a luer lug 34a extending circumferentially about the proximal end thereof, with an internal luer taper 36a which extends internally within a portion of hub housing 26a. Internal luer taper 36a is designed to engage a separate member, such as a medical device in the form of a hypodermic syringe or standard needle holder, or a handle portion, as described in more detail with respect to the embodiment of FIG. 7 described herein. Threads (not shown) may further be provided within internal luer taper 36a, for establishing threaded engagement with a corresponding threaded surface of such a separate member.

In the embodiment of FIG. 6, base 40a further includes a forward annular skirt 52a extending from distal end 46a of base 40a. Annular skirt 52a includes a tapered nub 56a having a profile for mating with the internal surface of internal luer taper 36a of hub 22a. In addition, annular skirt 52a includes internal threads 54a extending within annular skirt 52a on an internal surface thereof. Internal threads 54a of annular skirt 52a mate with luer lug 34a of hub 22a, thereby providing interengaging threaded structure between base 40a and hub 22a, for attaching unit dose needle 12a to base 40a. Alternatively, a system of luer locks may be used to effect mating between base 40a and hub 22a. Further, base 40a may include an internal luer taper 58a at the proximal end thereof, for further mating with a separate member such as a medical device or a handle assembly.

In such an embodiment including unit dose needle 12a and base 40a attached through hub 22a, the sleeve is provided in a similar manner as described above. A needle cover (not shown) may further be provided, which may engage tapered surface 32a of hub 22a, providing a sterile packaging cover for unit dose needle 12a during shipping and storage.

Such an embodiment enables unit does needle 12a and hub 22a to be provided as a separate member in the form of a unit dose assembly. Such a unit dose assembly can be mated with a separate shield assembly, including a base 40a with a sleeve extending thereabout, as described hereinabove. The unit dose assembly and the shield assembly are desirably attached, such as internal luer taper 36a and annular skirt 52a. In this manner, the unit dose needle assembly including unit dose needle 12a and hub 22a can be separately packaged as a sterile assembly (which may include a removable needle packaging cover,) and can be mated with the separate shield assembly immediately prior to use.

In another embodiment depicted in FIGS. 7 and 8, a base 40b includes a handle extension 60b, extending from a proximal end 44b of the base 40b. Handle extension 60b is designed to extend beyond the proximal end of the sleeve when a sleeve is provided around base 40b. As such, handle extension 60b provides base 40b with a surface for grasping by the user, thereby allowing the user to hold the base 40b in one hand and manipulate the sleeve with the other hand, to axially displace the sleeve with respect to base 40b and thereby shield or unshield unit dose needle 12b. Handle extension 60b can also function as a handle to facilitate administering a vaccine with unit dose needle 12b.

Handle extension 60b may be integrally formed with base 40b, or may be a separate member which is attached to the proximal end 44b of base 40b, such as through an internal luer taper as discussed with reference to FIG. 6. The handle extension 60b may include a profile for accommodating a user's fingers. For example, handle extension 60b may include one or more arcuate surfaces 62b desirably extending along opposing sides thereof, for grasping between a user's finger and thumb. Moreover, handle extension 60b may include an internal luer taper 64b, for further mating with a separate member such as a medical device.

In a further embodiment of the invention, the sleeve may further include a profile for accommodating a user's fingers. For example, as shown in FIG. 9, sleeve 80c may include a ribbed surface 98c formed through a plurality of ribs oriented circumferentially about housing 82c of sleeve 80c, which may extend over a portion of housing 82c or over the entire length of housing 82c. Ribbed surface 98c facilitates handling of the needle assembly by a user, whereby sleeve 80c can be manipulated or rotated about base 40c and moved axially with respect thereto through the guiding action of guide tab 50c within guide slot 90c, to place the needle assembly in an extended position or a retracted position. Although ribbed surface 98c is shown, other types of ergonomically acceptable surfaces to accommodate a user's fingers may be used, such as one or more arcuate surfaces, indentations, or bumps to facilitate gripping of the sleeve. Moreover, in such an embodiment, sleeve 80c can function as both a protective shield for unit dose needle 12c and as a handle to facilitate administering a vaccine with unit dose needle 12c.

FIGS. 10 and 11 depict a further embodiment of the present invention, in which the sleeve 80d includes a tab 102d extending preferably from an internal surface of the tubular housing 82d, for interengagement within a slot 70d extending along a portion of the outer surface of the housing 42d of base 40d. The slot 70d extends longitudinally along at least a portion of the length of the outer surface of housing 42d of base 40d. The sleeve 80d is capable of being slidably movable with respect to the base 40d and unit dose needle 12d to a position in which unit dose needle 12d is shielded, as shown in FIG. 11. During such slidable movement, tab 102d is guided within the slot 70d, thereby providing a guiding action that enables the unit dose needle 12d to be exposed and shielded between an extended position with the unit dose needle 12d extending from sleeve 80d, as shown in FIG. 10, and a retracted position with the unit dose needle 12d contained within the sleeve 80d, as shown in FIG. 11. A recessed pocket 72d may further be provided at a portion of the slot 70d adjacent the distal end of base 40d, which provides a means for locking the sleeve 80d in a position with the unit dose needle 12d retracted and shielded within sleeve 80d.

In such an embodiment, sleeve 80d may further include a profile for accommodating a user's fingers, such as one or more arcuate surfaces 100d.

In a further embodiment shown in FIG. 12, the base 40e includes latches 74e extending longitudinally along housing 42e from proximal end 44e toward distal end 46e, forming a clip end 76e. Such latches 74e are provided for releasable engagement with sleeve 80e, such as through detents 106e at proximal end 84e of sleeve 80e. The latches 74e engage the sleeve 80e in such a way as to retain the sleeve 80e in a first position with the unit dose needle 12e in the extended position. The latches 74e can be flexed to release the sleeve 80e, enabling the sleeve 80e to be moved to a position covering unit dose needle 12e. A biasing member such as a spring (not shown) may also be provided to automatically propel sleeve 80e to a position encompassing unit dose needle 12e upon release of the engagement between latches 74e and detents 106e. A compression spring (not shown) can be used to accomplish this function.

A further embodiment of the present invention is depicted in FIGS. 13 and 14, which includes a tip guard assembly 120f connected to base 40f through a flexible tether mechanism 110f. Tip guard assembly 120f may be provided as a one-component tip guard assembly, as depicted in FIGS. 13-14, or may be provided as a multiple-component assembly including a separate flexible tip guard attached within a housing. Tip guard assembly 120f including a housing 122f forming a top extent 124f defining the top portion of the tip guard assembly 120f and extending longitudinally along a portion of unit dose needle cannula 12f. Top extent 124f bends downwardly at a forward end to form front end wall 126f. At the rearward end, top extent 124f bends backward to form spring leg 128f which extends toward the front end wall 126f of tip guard assembly 120f, with lock out leg 130f bending upward and backward to form an end wall, as seen in FIG. 14.

Tip guard assembly 120f is interconnected with base 40f through tether mechanism 10f. Tether mechanism 10f includes a forward arm 112f and a rearward arm 114f. Forward arm 112f is attached to top extent 124f of tip guard assembly 120f and extends toward the rearward end of the needle assembly. Rearward arm 114f is attached to the top surface of housing 42f of base 40f, and also extends toward the rearward end of the needle assembly. Forward arm 112f and rearward arm 114f meet and are interengaged at pivot hinge 116f, providing a pivoting point therebetween. Additionally, forward arm 112f and rearward arm 114f are pivotally attached to tip guard assembly 120f and base 40f respectively such as through living hinge members 113f and 115f respectively. Forward living hinge member 113f connecting forward arm 112f to tip guard assembly 120f is preferably more rigid than rearward hinge member 115f such that movement of pivot hinge 116f by forward manual user exertion causes tip guard assembly 120f to move forward.

Tip guard assembly 120f is slidably movable in a telescoping fashion along unit dose needle 12f between a first position adjacent base 40f, and a second position encompassing prongs 18f of unit dose needle 12f through tether mechanism 110f. In particular, pivoting movement of forward arm 112f and rearward arm 114f about pivot hinge 116f, such as by forward pressure exerted thereon, causes a pivoting movement at pivot hinge 116f. This causes forward arm 112f to extend, thereby telescoping tip guard assembly 120f along unit dose needle 12f toward prongs 18f. During such movement, tip guard assembly 12f slides or glides along unit dose needle 12f, with lock out leg 130f passing distally beyond prongs 18f, and with the inherent resiliency of spring leg 128f urging lock out leg 130f over prongs 18f, preventing a return movement, and thus preventing re-exposure of prongs 18f.

Base 40f may further include handles 78f extending along opposing sides thereof, for facilitating holding of the assembly. In addition, base 40f may include an internal luer taper 36f for attachment to a separate medical device for providing further support for the user, as described above.

The sleeve, base, and hub of the assembly of the present invention may be constructed of any material, and are desirably constructed of a moldable plastic material. Suitable moldable plastics include, but are not limited to, polyethylenes, polypropylenes, polyamides, polyesters, and fluorinated polyethylenes.

While the present invention is satisfied by embodiments in many different forms, there is shown in the drawings and described herein in detail, the preferred embodiments of the invention, with the understanding that the present disclosure is to be considered as exemplary of the principles of the invention and is not intended to limit the invention to the embodiments illustrated. Various other embodiments will be apparent to and readily made by those skilled in the art without departing from the scope and spirit of the invention. The scope of the invention will be measured by the appended claims and their equivalents.

What is claimed is:

1. A shieldable, single-use needle assembly for administering a unit dose of a vaccine comprising:
(a) a sleeve including a tubular wall defining an internal sleeve opening extending between a proximal end and an opposed distal end, said sleeve including a guide slot extending through the tubular wall into the internal sleeve opening, said guide slot including a longitudinal extent extending longitudinally along the sleeve, a proximal limit extent disposed at a first end of the guide slot adjacent said proximal end of said tubular wall and a distal limit extent disposed at a second end of the guide slot adjacent said distal end of said tubular wall, said proximal and distal limit extents extending along a circumferential surface of the tubular wall;
(b) a base including a tab disposed on an outer surface thereof, said base contained within said internal sleeve opening with said tab extending through and beyond said guide slot; and
(c) a unit dose needle having a base end attached to the base and an opposed prong end configured to hold a unit dose of a vaccine;
wherein movement of the tab through the guide slot between the proximal limit extent and the distal limit extent provides a guiding action that enables an extension and a retraction movement of the needle between an extended position with the needle extending from the distal end of the sleeve and a retracted position with the needle contained within the internal sleeve opening, and wherein the proximal and distal limit extents fix the position of the needle between the extended position and the retracted position.

2. The needle assembly of claim 1, wherein the unit dose needle comprises a bifurcated needle, wherein the prong end includes two pointed prongs which are capable of penetrating or abrading the skin of a patient, and wherein the prongs are separated by a U-shaped channel capable of holding the unit dose of a vaccine.

3. The needle assembly of claim 1, wherein the unit dose of a vaccine is a liquid.

4. The needle assembly of claim 1, wherein the base includes a handle extension extending beyond the sleeve from an end of the base opposite the unit dose needle.

5. The needle assembly of claim 4, wherein the handle extension includes an internal luer taper to engage a medical device.

6. The needle assembly of claim 4, wherein the handle extension includes a profile for accommodating a user's fingers.

7. The needle assembly of claim 6, wherein the handle extension includes one or more arcuate surfaces.

8. The needle assembly of claim 1, further comprising a hub fixedly attached to the base end of the needle for attaching the needle to the base.

9. The needle assembly of claim 8, wherein the base and the hub include corresponding engaging surfaces for attachment therebetween.

10. The needle assembly of claim 9, wherein the base and the hub include corresponding engaging surfaces for threaded engagement therebetween.

11. The needle assembly of claim 1, wherein the sleeve comprises one or more moldable plastics.

12. The needle assembly of claim 11, wherein the plastics are one or more selected from the group consisting of polyethylenes, polypropylenes, polyamides, polyesters and fluorinated polyethylenes.

13. A shieldable, single-use needle assembly for administering a unit dose of a vaccine comprising:
(a) a sleeve including a tubular wall defining an internal sleeve opening extending between a proximal end and an opposed distal end, said sleeve including a guide slot extending through the tubular wall into the internal sleeve opening, said guide slot including a longitudinal extent extending longitudinally along the sleeve, a proximal limit extent disposed at a first end of the guide slot adjacent said proximal end of said tubular wall and a distal limit extent disposed at a second end of the guide slot adjacent said distal end of said tubular wall, said proximal and distal limit extents extending along a circumferential surface of the tubular wall;
(b) a base including a tab disposed on an outer surface thereof, said base slidably engaged in said internal sleeve opening with said tab extending through said guide slot, said base including a handle extension extending beyond the proximal end of the sleeve; and
(c) a unit dose needle having a base end attached to the base and an opposed prong end configured to hold a unit dose of a vaccine;
wherein the shield is slidably movable about the needle assembly between a first position in which the needle extends from the distal end of the sleeve and a second position in which the needle is contained within the internal sleeve opening, the tab and the guide slot providing for guiding action through the guide slot between the proximal limit extent and the distal limit extent, thereby causing the shield to move about the needle assembly between the first position and the second position.

14. The needle assembly of claim 13, wherein the proximal and distal limit extents fix the position of the needle between the first position and the second position, respectively.

15. The needle assembly of claim 13, wherein the handle extension includes an internal luer taper to engage a medical device.

16. The needle assembly of claim 13, wherein the handle extension includes a profile for accommodating a user's fingers.

17. The needle assembly of claim 17, wherein the handle extension includes one or more arcuate surfaces.

18. A shieldable single-use needle assembly for administering a unit dose of a vaccine comprising:
(a) a unit dose needle having a prong end configured to hold a unit dose of a vaccine;
(b) a hub attached to the unit dose needle opposite the prong end, the hub including a tapered luer;
(c) a base including a tab disposed on an outer surface thereof, said base threadably attached to the tapered luer of the hub; and
(d) a hollow sleeve extending about said base, said sleeve including a tubular wall extending between a proximal end and an opposed distal end and defining an internal sleeve opening, said sleeve including a guide slot extending through the tubular wall into the internal sleeve opening, said guide slot including a longitudinal extent extending longitudinally along the sleeve, a proximal limit extent disposed at a first end of the guide slot adjacent said proximal end of said tubular wall and a distal limit extent disposed at a second end of the guide slot adjacent said distal end of said tubular wall, said proximal and distal limit extents extending along a circumferential surface of the tubular wall;
wherein said tab extends through and beyond said guide slot such that movement of the tab through the guide slot between the proximal limit extent and the distal limit extent provides a guiding action that enables an extension and a retraction movement of the needle between an extended position with the needle extending from the distal end of the sleeve and a retracted position with the needle contained within the internal sleeve opening, and wherein the proximal and distal limit extents fix the position of the needle between the extended position and the retracted position.

19. A shieldable needle assembly for administering a unit dose of a vaccine comprising:
a) a unit dose needle assembly comprising:
i) a unit dose needle including a prong end configured to hold a unit dose of a vaccine, and
ii) a hub fixedly attached to the unit dose needle opposite the prong end; and
b) a shield assembly attached to said unit dose needle assembly, said shield assembly comprising:
i) a sleeve including a tubular wall defining an internal sleeve opening extending between a proximal end and an opposed distal end, said sleeve including a guide slot extending through the tubular wall into the internal sleeve opening; and
ii) a base including a tab disposed on an outer surface thereof, said base contained within said internal sleeve opening with said tab extending through and beyond said guide slot;
wherein the hub of the unit dose needle assembly and the base of the shield assembly include corresponding structure for attachment therebetween, and wherein the sleeve and the base are movable with respect to each other, thereby enabling an extension and a retraction movement of the needle between an extended position with the needle extending from the distal end of the sleeve and a retracted position with the needle contained within the internal sleeve opening.

20. The needle assembly of claim 19, wherein the hub and the base include corresponding threaded surfaces for threaded engagement therebetween.

21. A shieldable, single-use needle assembly for administering a unit dose of a vaccine comprising:
(a) a base;
(b) a unit dose needle having a base end attached to the base and an opposed prong end configured to hold a unit dose of a vaccine;
(c) a sleeve including a tubular wall defining an internal sleeve opening extending between a proximal end and an opposed distal end, said sleeve axially coacting with said base between a prong exposed position and a prong encapsulated position; and
(d) a guiding element interengaged between said sleeve and said base providing limited axial movement of said sleeve with respect to said base.

22. The needle assembly of claim 21, wherein said guiding element is a tab extending from said base into a slot in said sleeve.

23. The needle assembly of claim 22, wherein said slot extends through said sleeve and said tab is a means to manually move said base unit dose needle into said sleeve.

24. The needle assembly of claim 21, wherein said base includes a handle extension extending beyond the proximal end of said sleeve.

25. A shieldable, single-use needle assembly for administering a unit dose of a vaccine comprising:
(a) a base;
(b) a unit dose needle having an axis, a base end attached to the base and an opposed prong end configured to hold a unit dose of a vaccine;
(c) a tip guard slidable along said unit dose needle axis from a first position adjacent said base to a second position encapsulating said unit dose needle prong end; and
(d) a tether element having a base end integral with said base, a guard end integral with said tip guard, a hinge between said base end and said guard end, a forward member delimited by said guard end and said hinge flexibly pivotable with respect to said guard, a rearward end delimited by said base end and said hinge flexibly pivotable with respect to said base, and an angle between said forward and rearward members wherein forward movement of said hinge enlarges said angle and provides direct forward movement of said guard with respect to said base.

26. The single-use needle assembly of claim 25, wherein said tip guard has a lockout leg preventing a return movement of said tip guard with respect to said base once said lockout leg moves past said prong end of said unit dose needle.
